(19) United States
(12) Patent Application Publication
(10) Pub. No.: US 2022/0409960 A1
(43) Pub. Date: Dec. 29, 2022

(54) BALANCE TRAINING SYSTEMS AND METHODS

(71) Applicants: University of Florida Research Foundation, Inc., Gainesville, FL (US); THE REGENTS OF THE UNIVERSITY OF MICHIGAN OFFICE OF TECHNOLOGY TRANSFER, Ann Arbor, MI (US)

(72) Inventors: Daniel Perry Ferris, Gainesville, FL (US); Evangelia-Regkina Symeonidou, Gainesville, FL (US); Steven Michael Peterson, Seattle, WA (US)

(21) Appl. No.: 17/623,920

(22) PCT Filed: Jun. 30, 2020
(86) PCT No.: PCT/US2020/040229

§ 371 Date: Dec. 30, 2021

Related U.S. Application Data
(60) Provisional application No. 62/869,778, filed on Jul. 2, 2019.

(51) Int. Cl. A63B 26/00; A63B 71/06; A63B 22/02; A63B 24/00

(52) U.S. Cl. CPC A63B 26/003; A63B 71/0622; A63B 22/02; A63B 24/0062; A63B 2214/00; A63B 69/0064

(57) ABSTRACT

Systems and methods are disclosed for human balance training. The systems and methods involve intermittently occluding the vision of a subject while the subject is engaged in a balance activity, such as walking on a balance beam, for example. Creating a brief period of continuous visual occlusion results in posterior parietal cortex activation, leading to enhanced balance training. The end result is much greater improvement in balance compared to physical training alone.

### CROSS-REFERENCE TO RELATED APPLICATIONS

This U.S. nonprovisional application claims priority to, and the benefit of the filing date of, U.S. provisional application No. 62/869,778, filed on Jul. 2, 2019, entitled “BALANCE TRAINING SYSTEMS AND METHODS,” which is incorporated herein by reference in its entirety.

### TECHNICAL FIELD

The present disclosure relates to systems and methods for balance training in human beings.

### BACKGROUND

Falls are a major health concern amongst multiple populations. Individuals with sensorimotor or musculoskeletal deficits, such as spinal cord injury or lower limb amputation, fall at greater rates than physically intact individuals. Falls are also prominent in the elderly and can have substantial socioeconomic consequences. There is a great need to identify better balance training protocols that are simple enough to be performed at home and that lead to reductions in falls.

Known or proposed balance training protocols typically focus on the use of visual, proprioceptive, or vestibular perturbations. Visual perturbations may be the easiest to enact without complicated equipment and vision is especially important when walking on a narrow surface or acquiring a new motor skill.

In one known study, participants trained while wearing a virtual reality headset. Some of the participants were subjected to visual rotations during the training and some of the participants trained without being subjected to visual rotations. The study results showed that the participants who were trained with visual rotations improved their balance performance four times as much as participants who trained without visual rotations. High-density electroencephalography data revealed that the visual rotations produced a strong response in the posterior parietal cortex during training. The rotation caused a strong synchronization in the theta and alpha frequency bands, followed by a strong desynchronization in the beta frequency band. It is possible that this stimulation of the posterior parietal cortex caused by the visual rotation led to the enhanced motor learning gains in balance control. Virtual reality headsets are, however, bulky, expensive, and induce motion sickness.

A need exists for a balance training protocol that can be implemented at home without the need for virtual reality headsets, that can be implemented at relatively low cost and that is very effective.

### SUMMARY

The present disclosure discloses balance training systems and methods. The system for training balance in a human subject comprises a human balance training course to be performed by the human subject during a balance training session, a pair of occlusion glasses to be worn by the human subject during the balance training session, one or more sensors configured to monitor the human subject as the human subject performs the human balance training course, a memory device, and a processor configured to perform a balance training algorithm. The occlusion glasses are configured to intermittently occlude the vision of the human subject as the human subject performs the human balance training course. The sensors that monitor the human subject as the human subject performs the human balance training course generate output signals corresponding to the performance by the human subject of the human balance training course. The processor performing the balance training algorithm processes the output signals to record the performance of the human subject in the memory device.

In accordance with an embodiment, the balance training algorithm processes the output signals to assess the performance of the human subject. In accordance with an embodiment, the balance training algorithm adjusts one or more of the timing, frequency and duration of the intermittent occlusions based at least in part on the assessment of the performance of the human subject. In accordance with an embodiment, the balance training algorithm records the assessment of the performance of the human subject in the memory device. In accordance with an embodiment, the balance training algorithm compares the recorded assessment with a previously recorded assessment of a performance by the human subject of the balance training course to determine whether the balance of the human subject has improved. In accordance with an embodiment, the previously recorded assessment was obtained from output signals generated by the one or more sensors monitoring the performance by the human subject of the balance training course while not wearing the pair of occlusion glasses and not being subjected to intermittently occluded vision. In accordance with an embodiment, the previously recorded assessment was obtained from output signals generated by the one or more sensors monitoring the performance by the human subject of the balance training course while wearing the pair of occlusion glasses and being subjected to intermittently occluded vision.

In accordance with an embodiment, the pair of occlusion glasses are incorporated into a housing in which the processor and memory device are housed. The housing comprises a user interface that has one or more controls that allow at least one of the timing, frequency and duration of the intermittent occlusions to be set and adjusted by a user.

The method for training balance in a human subject comprises:
providing a human balance training course to be performed by the human subject during a balance training session during which the human subject wears a pair of occlusion glasses during the balance training session;
intermittently occluding the vision of the human subject during the balance training session by switching the occlusion glasses between a clear state in which the vision of the human subject is unoccluded and an occluded state in which the vision of the human subject is occluded, and vice versa;
with one or more sensors, monitoring the human subject during the balance training session and generating output signals corresponding to the performance by the human subject during the balance training session; and
with a processor, performing a balance training algorithm that processes the output signals and records the performance of the human subject in a memory device.

In accordance with an embodiment, he method further comprises, with the processor performing the balance training algorithm, processing the output signals to assess the performance of the human subject.

In accordance with an embodiment, the method further comprises, with the processor performing the balance training algorithm, causing one or more of a timing, frequency and duration of the intermittent occlusions to be adjusted based at least in part on the assessment of the performance of the human subject.

In accordance with an embodiment, the method further comprises, with the processor performing the balance training algorithm, recording the assessment of the performance of the human subject in the memory device. In accordance with an embodiment, the method further comprises, with the processor performing the balance training algorithm, comparing the recorded assessment with a previously recorded assessment of a performance by the human subject of the balance training course to determine whether the balance of the human subject has improved. In accordance with an embodiment, the previously recorded assessment was obtained from output signals generated by said one or more sensors monitoring the performance by the human subject of the balance training course while not wearing the pair of occlusion glasses and not being subjected to intermittently occluded vision.

In accordance with an embodiment, the previously recorded assessment was obtained from output signals generated by said one or more sensors monitoring the performance by the human subject of the balance training course while wearing the pair of occlusion glasses and being subjected to intermittently occluded vision.

In accordance with an embodiment, the pair of occlusion glasses are incorporated into a housing in which the processor and memory device are housed. The housing comprises a user interface that has one or more controls that allow at least one of a timing, frequency and duration of the intermittent occlusions to be set and adjusted by a user.

In accordance with an embodiment, a computer program comprises computer instructions for execution by a processor. The computer instructions are embodied on a non-transitory computer-readable medium. The computer instructions comprise:

a first set of computer instructions for receiving output signals output from one or more sensors that monitor a performance by a human subject of balance training course during a balance training session during which the human subject wears a pair of occlusion glasses that intermittently occlude the vision of the human subject during the balance training session by switching the occlusion glasses between a clear state in which the vision of the human subject is unoccluded and an occluded state in which the vision of the human subject is occluded, and vice versa; and

a second set of computer instructions that process the output signals to record the performance of the human subject in a memory device.

In accordance with an embodiment, the computer program further comprises a third set of computer instructions that process the output signals to assess the performance of the human subject.

In accordance with an embodiment, the computer program further comprises a fourth set of computer instructions that cause one or more of the timing, frequency and duration of the intermittent occlusions to be adjusted based at least in part on the assessment of the performance of the human subject.

In accordance with an embodiment, the computer program further comprises a fifth set of computer instructions that compare the recorded assessment with a previously recorded assessment of a performance by the human subject of the balance training course to determine whether the balance of the human subject has improved.

These and other features and advantages will become apparent from the following description, drawings and claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a side view of a training participant walking on a treadmill-mounted balance beam of the system while wearing occlusion glasses of the system in accordance with a representative embodiment.

FIG. 2 is a block diagram of a data processing unit of the system in accordance with a representative embodiment for recording and assessing balance training performance.

FIG. 3 is a bar graph demonstrating balance improvement for an unperturbed vision group and for a visual occlusions group measured on the same day and two weeks after the training for a first study; the bar graph demonstrates training with brief, intermittent visual occlusions substantially boosted the training effect of beam walking practice.

FIG. 4 is a bar graph demonstrating the balance improvement for an unperturbed vision group and for a visual occlusions group measured on the same day and two weeks after the training for a second study; the bar graph demonstrates that training with brief, intermittent visual occlusions substantially boosted the training effect of beam walking practice.

FIGS. 5A and 5B show cortical cluster centroids in the sagittal and coronal views of an electroencephalogram (EEG) for eight out of the ten individuals whose vision was intermittently occluded in the second study.

FIGS. 6A-6D are event-related spectral perturbation plots (ERSPs) epoched around the occlusion onset (0 ms) and offset (1500 ms) for one of the posterior parietal (FIG. 6A), the occipital (FIG. 6B), the temporal (FIG. 6C), and the sensorimotor clusters (FIG. 6D) for eight out of the ten participants of the visual occlusions group in the second study.

FIGS. 7A-7D are spectral power plots before and after the training for the visual occlusions group in the second study for the posterior parietal, occipital, temporal, and sensorimotor clusters, respectively.

FIG. 8 is a flow diagram representing the method in accordance with a representative embodiment.

FIG. 9 is a flow diagram representing the method in accordance with a representative embodiment.

FIG. 10 is a flow diagram representing the method in accordance with a representative embodiment.

### DETAILED DESCRIPTION

The present disclosure discloses representative, or exemplary, embodiments of systems and methods for balance training. The system comprises a human balance training course to be performed by a human subject participating in a balance training session, a pair of occlusion glasses to be worn by the human subject during the balance training session that intermittently occlude vision of the human subject as the human subject performs the human balance training course, one or more sensors configured to monitor the human subject during the balance training session and to output signals corresponding to the monitored performance, and a data processing unit configured to process the output signals to record the performance and to assess the performance. The system can be simple enough to be used at home at relatively low cost and is very effective at improving balance, thereby leading to great reductions in falls.

In the following detailed description, for purposes of explanation and not limitation, exemplary, or representative, embodiments disclosing specific details are set forth in order to provide a thorough understanding of an embodiment according to the present teachings. However, it will be apparent to one having ordinary skill in the art having the benefit of the present disclosure that other embodiments according to the present teachings that depart from the specific details disclosed herein remain within the scope of the appended claims. Moreover, descriptions of well-known apparatuses and methods may be omitted so as to not obscure the description of the example embodiments. Such methods and apparatuses are clearly within the scope of the present teachings.

The terminology used herein is for purposes of describing particular embodiments only and is not intended to be limiting. The defined terms are in addition to the technical and scientific meanings of the defined terms as commonly understood and accepted in the technical field of the present teachings.

As used in the specification and appended claims, the terms “a,” “an,” and “the” include both singular and plural referents, unless the context clearly dictates otherwise. Thus, for example, “a device” includes one device and plural devices.

Relative terms may be used to describe the various elements' relationships to one another, as illustrated in the accompanying drawings. These relative terms are intended to encompass different orientations of the device and/or elements in addition to the orientation depicted in the drawings.

It will be understood that when an element is referred to as being “connected to” or “coupled to” or “electrically coupled to” another element, it can be directly connected or coupled, or intervening elements may be present.

The term “memory” or “memory device”, as those terms are used herein, are intended to denote a non-transitory computer-readable storage medium that is capable of storing computer instructions, or computer code, for execution by one or more processors. References herein to “memory” or “memory device” should be interpreted as one or more memories or memory devices. The memory may, for example, be multiple memories within the same computer system. The memory may also be multiple memories distributed amongst multiple computer systems or computing devices.

A “processor”, as that term is used herein encompasses an electronic component that is able to execute a computer program or executable computer instructions. References herein to a computer comprising “a processor” should be interpreted as one or more processors or processing cores. The processor may for instance be a multi-core processor. A processor may also refer to a collection of processors within a single computer system or distributed amongst multiple computer systems. The term “computer” should also be interpreted as possibly referring to a collection or network of computers or computing devices, each comprising a processor or processors. Instructions of a computer program can be performed by multiple processors that may be within the same computer or that may be distributed across multiple computers.

It is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

Exemplary, or representative, embodiments will now be described with reference to the figures, in which like reference numerals represent like components, elements or features. It should be noted that features, elements or components in the figures are not intended to be drawn to scale, emphasis being placed instead on demonstrating inventive principles and concepts.

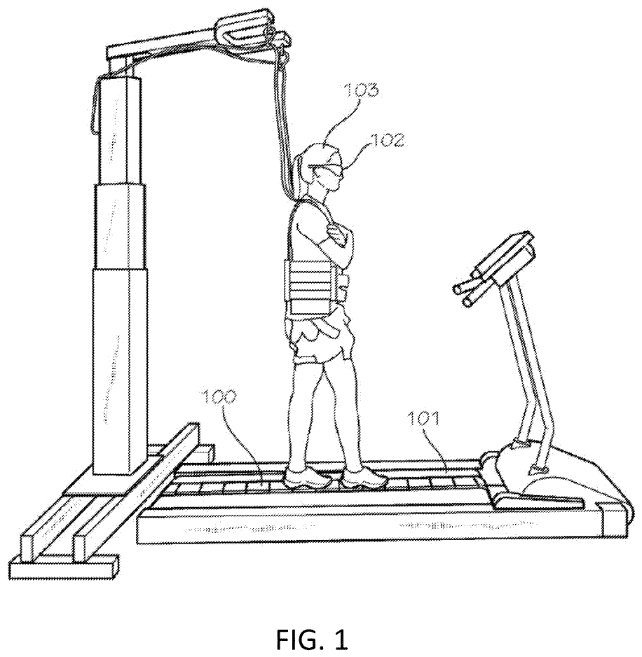

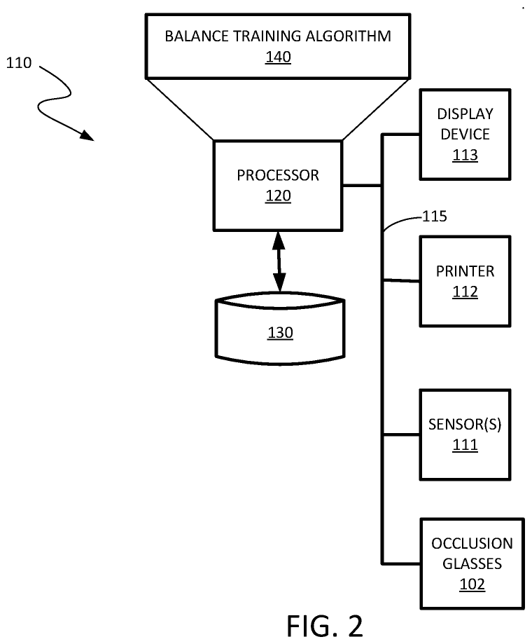

FIG. 1 is a side view of a human subject performing a human balance training course while wearing occlusion glasses in accordance with a representative embodiment in which the human balance training course comprises a balance beam 100 mounted on a treadmill 101. In accordance with this representative embodiment, the system comprises the human balance training course 100, 101, a pair of intermittent googles 102 worn by a human subject 103, one or more sensors (not shown) and a data processing unit (not shown). The sensor(s) and the data processing unit will now be described with reference to FIG. 2.

FIG. 2 is a block diagram of the data processing unit 110 in accordance with a representative embodiment. The data processing unit 110 comprises a processor 120 and a memory device 130. The processor 120 is configured to perform a balance training algorithm 140 that receives the output signals generated by the sensor(s) 111 and processes the output signals to record and assess the performance of the human subject 103. In some embodiments, the balance training algorithm 140 also controls the occlusion glasses 102 to switch them between a clear state and an occluded state, and vice versa. In other embodiments, the occlusion glasses 102 are pre-configured to switch between the clear state and the occluded state, and vice versa, in accordance with a predetermined switching algorithm, and therefore no intervention from the processor 120 is needed. The glasses 102 may have a processor that can be configured to cause the glasses to switch between the clear state and the occluded state with preselected intermittency of the occluded state.

In accordance with a preferred embodiment, the balance training algorithm 140 controls the occlusion glasses 102. For this embodiment, the connection between the processor 120 and the occlusion glasses 102 can be a wired connection or a wireless connection. Likewise, the connection between the processor 120 and the sensor(s) 111 can be a wired connection or a wireless connection. The memory device 130 typically stores computer instructions for execution by the processor 120 and data corresponding to the recorded performance and the assessment of the performance. In accordance with the embodiment, the algorithm 140 activates the occlusion glasses 102 and controls the timing, frequency and/or duration of occlusion. In accordance with a representative embodiment, the algorithm 140 adjusts the timing, frequency and/or duration of occlusion based at least in part on the frequency at which the subject steps off of the beam 100.

In other embodiments, the occlusion glasses 102 has its own processor or controller that can be programmed or configured to allow the timing, frequency and/or duration of occlusion to be set. In such embodiments, the processor or controller of the occlusion glasses 102 can be programmed or configured to adjust the timing, frequency and/or duration of occlusion based at least in part on the frequency at which the subject steps off of the beam 100. The data processing unit 110 may include other optional devices, such as, but not limited to, a printer 112 and a display device 113 for printing and displaying the recorded data and/or performance assessments, respectively. The sensor(s) 111, the printer 112 and the display device 113 may be connected to a bus 115 to which the processor 120 is also connected to allow the processor 120 to control operations performed by those devices. However, while the bus 115 is depicted as a wired connection in FIG. 2, one or more of these devices may communicate via one or more wireless links.

In the following examples, the system shown in FIGS. 1 and 2 was used to provide balance training to multiple individuals whose performances were recorded and assessed.

### EXAMPLES

### Example 1: Training with Brief Visual Occlusions Improves Balance Control in Treadmill Beam Walking

In accordance with the inventive principles and concepts of the present disclosure, as the human subject performs the human balance training course, the vision of the human subject is occluded at times and is unoccluded at times by placing the glasses 102 in the occluded state or the clear state, respectively. The occlusion glasses 102 can be off-the-shelf glasses. For example, Senaptec of Oregon, USA sells occlusion glasses that are suitable for this purpose. Such glasses were used in the current study to introduce transient visual perturbations while participants walked on the treadmill-mounted balance beam 100. The glasses 102 had the ability to change from clear to opaque, restricting the participant's vision. The studies demonstrated that training participants with brief visual occlusions leads to an increase in balance performance compared to training participants with unperturbed vision, and that this effect can be retained for at least two weeks after the initial training.

First Study

Twenty healthy young adults were randomly assigned to one of two training protocols: a) training with visual occlusions (6 females, 4 males); and b) training with unperturbed vision (5 females, 5 males). During the balance training sessions for the group a) training protocol, each participant was presented with periodic 1.5 second visual occlusions followed by 7.5 seconds of clear vision. During the balance training sessions for the group b) training protocol, each participant performed the training retaining their full vision at all times. The training took place on the treadmill-mounted balance beam 100 shown in FIG. 1, which was 2.5 cm high and 2.5 cm wide. It should be noted that the present disclosure is not limited to the human balance training course being a treadmill-mounted balance beam. The human balance training course can be anything that provides suitable human balance training conditions.

To evaluate changes in performance, participants performed 3-minute pre-tests, post-tests and retention tests. During these tests, subjects walked on the beam 100 without any visual perturbations. The post-test took place on the same day as the balance training session. The retention test took place two weeks later. Performance was evaluated by counting the number of step-offs per minute, i.e., the number of times the participant stepped off of the beam per minute. Balance improvement was quantified as the difference between the pre-test and post-test performance (same day), and the difference between the pre-test and retention test performance (two weeks later). To normalize across different subject skill levels, the balance improvement metrics were divided by the pre-test performance. It should be noted that vision preferably is not occluded during the pre-tests, post-tests or retention tests when assessing balance improvement, but during balance training.

Repeated analysis of variances (ANOVA) tests were performed with the training protocol (visual occlusions, unperturbed vision) as the between-subjects variable and the testing day (same day, two weeks later) as the within-subjects variable. Independent t-tests were also performed between the two groups for each day, to evaluate performance differences for both time points.

Results of First Study

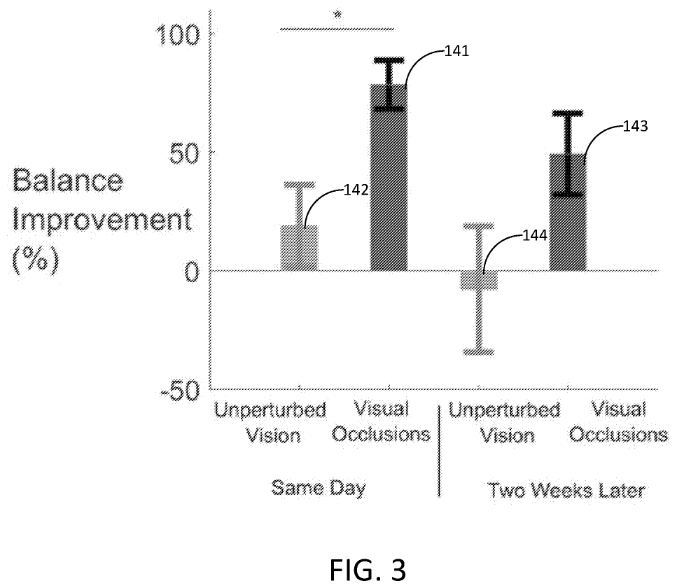

FIG. 3 is a bar graph demonstrating the balance improvement for the unperturbed vision group (group b)) and for a visual occlusions group (group a)) measured on the same day and two weeks after the training. The bar graph demonstrates training with brief, intermittent visual occlusions substantially boosted the training effect of beam walking practice. Training with brief intermittent occlusions had a significant effect on balance improvement compared to training without occlusions (F(1,18)=7.2, p<0.05). The F ratio here and the p value prove that the intermittent occlusions have significant effect on balance performance improvement, which is also apparent from FIG. 3. It can be seen from a comparison of bars 141 and 142 that the visual occlusions group showed a four times higher improvement compared to the unperturbed vision group on the same day (t(18)=−2.95, p<0.01). Bar 143 shows that the visual occlusions group retained the majority of the training effect two weeks later (49% performance improvement). Bar 144 shows that the control group that trained without occlusions showed no retention of the training improvement.

In a subsequent experiment discussed below, electrocortical changes that occur with the visual occlusion are quantified. As indicated above, visual rotations resulted in activation of occipital and posterior parietal areas, with a pattern of alpha/theta synchronization and beta desynchronization following the rotation onset and offset. It is believed that the brief, intermittent visual occlusion in accordance with the present disclosure also produce a similar stimulation of the occipital and posterior parietal cortex. The parietal cortex plays a key role in cross modal integration. It receives and enhances information from different modalities relevant to a task. It has already been shown in macaque monkeys that changing the content of visual information can induce reweighting of different sensory modalities. It is possible that introducing visual perturbations shifted the weighting from the visual to the proprioceptive and vestibular channels, resulting in performance enhancement and long-term motor learning. Understanding the underlying neurophysiological responses caused by visual perturbations should help design better training paradigms.

The current study discussed herein only included healthy young adults that were physically intact. Based on the current results, it would seem reasonable that testing patient and elderly populations might also reveal enhanced balance training effects with visual occlusion.

Second Study

In the second study, the balance training involved having a total of forty healthy young individuals walk for thirty minutes on the treadmill-mounted balance beam 100. Their performances were recorded and their performance improvements were assessed on the same day of the training and two weeks later. Twenty of the individuals trained wearing the occlusion glasses 102 that induced repetitive brief visual occlusions and twenty of the individuals trained with no visual occlusions. To look into changes in brain activity during and after the training, high-density EEG recordings were obtained on ten of the participants of the visual occlusions group. The inventors expected spectral activity modulations in regions involved in gait and balance control such as the posterior parietal, occipital, temporal, and sensorimotor cortex during training. The inventors also expected spectral activity changes after the training.

Results of Second Study

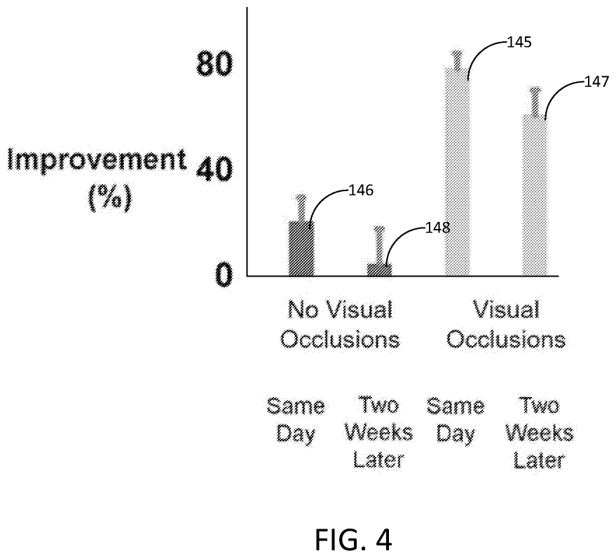

FIG. 4 is a bar graph demonstrating the balance improvement for the unperturbed vision group and for a visual occlusions group measured on the same day and two weeks after the training. The bar graph demonstrates that training with brief, intermittent visual occlusions substantially boosted the training effect of beam walking practice. A comparison of bars 145 and 146 show that balance performance improved by ˜80% for the visual occlusions group (bar 145) on the same day of the training, a four-fold improvement compared to the no visual occlusions group (bar 146). Forty percent of participants that trained with visual occlusions experienced absolutely no falls after training. Also, individuals of the visual occlusions group retained 75% of their performance improvement two weeks later, as indicated by bar 147, compared to only about 5% of the unperturbed vision group, as indicated by bar 148.

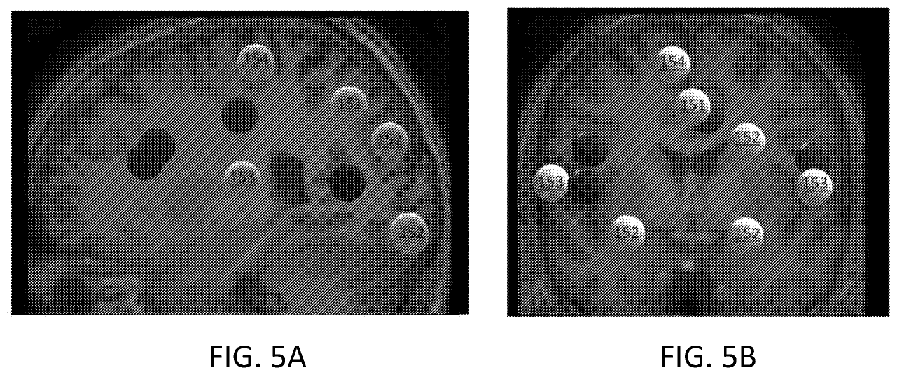

FIGS. 5A and 5B show cortical cluster centroids in the sagittal and coronal views of the EEG for eight out of the ten individuals whose vision was intermittently occluded. Clusters 151, 152, 153 and 154 correspond, respectively, to the posterior parietal cluster, the occipital clusters, the temporal clusters and the sensorimotor cluster. As expected by the inventors, the EEG analysis revealed activation in brain regions related to gait and balance.

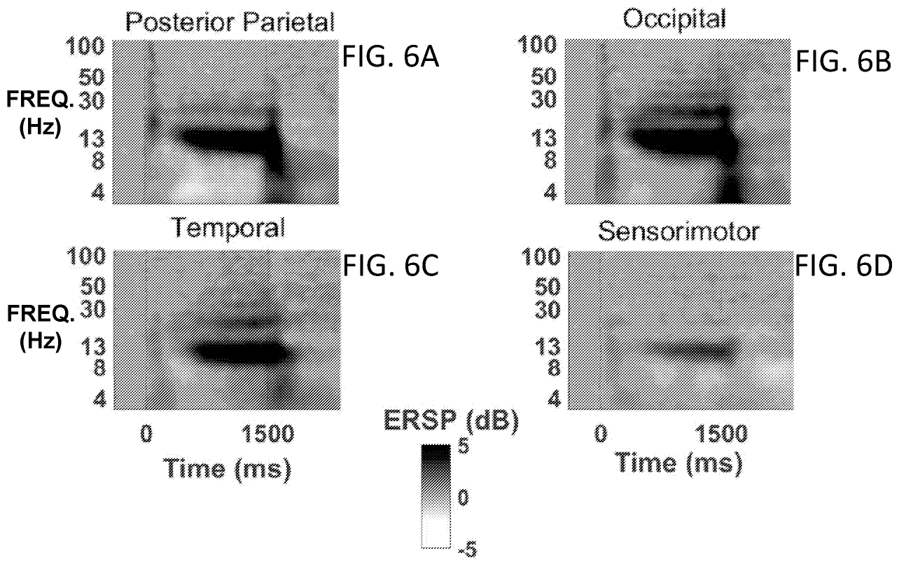

FIGS. 6A-6D are event-related spectral perturbation plots (ERSPs) epoched around the occlusion onset (0 ms) and offset (1500 ms) for one of the posterior parietal (FIG. 6A), the occipital (FIG. 6B), the temporal (FIG. 6C), and the sensorimotor clusters (FIG. 6D) for eight out of the ten participants of the visual occlusions group. Non-significant ERSP power was set to 0. Synchronization is depicted in the darker regions and desynchronization is depicted in lighter regions. Specifically, alpha (8-13 Hz) synchronization was observed in the posterior parietal, occipital, temporal, and sensorimotor cortex after the occlusion onset. The posterior, occipital, and temporal cortex also exhibited alpha and theta (6-8 Hz) synchronization after vision was restored. The occipital and temporal cortex exhibited additional beta (13-32 Hz) synchronization 500 ms after the occlusion onset. There were also changes in spectral power before and after the training in the above-mentioned brain regions. The posterior parietal, temporal and sensorimotor cortex exhibited an increase in alpha power after the occlusion training, while the occipital cortex showed a decrease in alpha power.

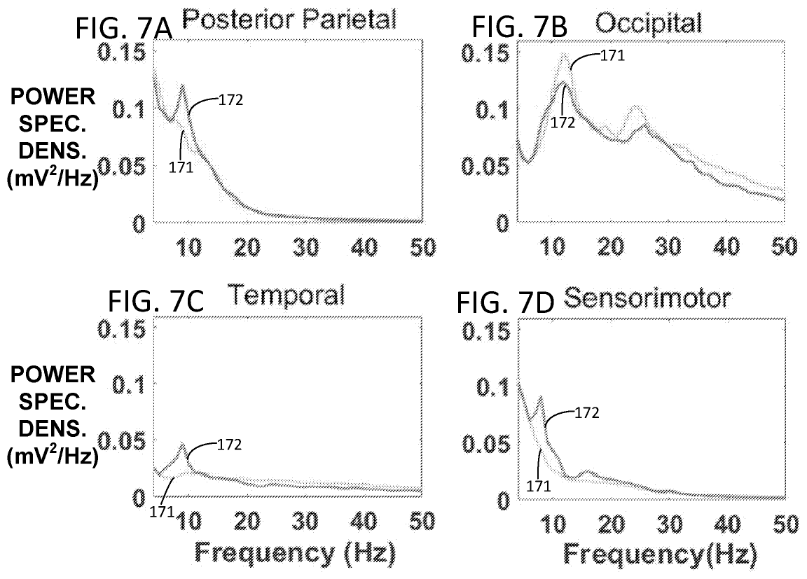

FIGS. 7A-7D are spectral power plots before and after the training for the visual occlusions group for the posterior parietal, occipital, temporal, and sensorimotor clusters, respectively. Average EEG spectral power for one of the posterior parietal, occipital, temporal, and sensorimotor clusters before (curve 171) and after (curve 172) the training for eight out of ten participants of the visual occlusions group.

The changes in brain activity in the posterior parietal, occipital, temporal, and sensorimotor cortex after the occlusion onset and offset, likely reflect activity modulations in these areas due to the occlusion. The changes in power in these regions after the training, suggest a processing shift from the occipital (visual) to the temporal (vestibular) and sensorimotor cortex, mediated by the posterior parietal cortex (sensory integrator), which possibly led to the long-term balance improvement. Thus, in accordance with the inventive principles and concepts, training intervention for dynamic balance control can be achieved, thereby leading to a substantial reduction of falls and long-term balance enhancement in a healthy population. This training intervention can be easily implemented in, for example, senior centers, rehabilitation centers, or at home. It can improve the quality of life of elderly, neurologically impaired individuals, or individuals recovering from injuries, or even help athletes that wish to improve their balance control.

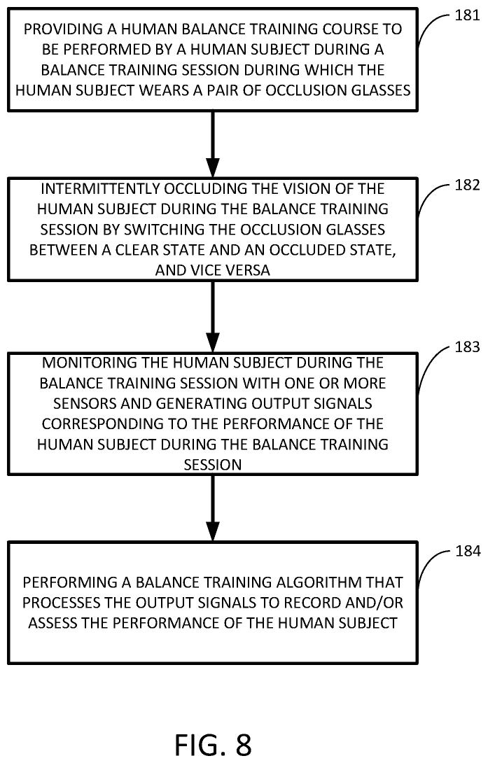

FIG. 8 is a flow diagram representing the method performed by the data processing unit 110 running the algorithm 140 in accordance with a representative embodiment. Block 181 represents the step of providing a human balance training course to be performed by a human subject during a balance training session during which the human subject wears a pair of occlusion glasses. Block 182 represents intermittently occluding the vision of the human subject during the balance training session by switching the occlusion glasses between a clear state in which the vision of the human subject is unoccluded and an occluded state in which the vision of the human subject is occluded, and vice versa. Although the inventive principles and concepts are not limited with regard to the durations of the clear state or occluded state, it has been determined through experimentation that the duration of the clear state should be long relative to the duration of the occluded state.

For example, the ratio of the duration of the clear state, T_{Clear}, to the duration of the occluded state, T_{Occluded}, or T_{Clear}/T_{Occluded}, is typically greater than or equal to 2 and less than or equal to 10:

2≤T_{Clear}/T_{Occluded}≤10

In the first study described above, during each training session, each participant was presented with periodic 1.5 second visual occlusions followed by 7.5 seconds of clear vision, which corresponds to T_{Clear}/T_{Occluded}, ratio of 7.5/1.5=5. As demonstrated above, this resulted in significant improvements in balance and significant retention in balance improvement. The ratio is typically in the range of:

3≤T_{Clear}/T_{Occluded}≤7

Block 183 represents monitoring the human subject during the balance training session with one or more sensors that generate output signals corresponding to the performance by the human subject during the balance training session. The inventive principles and concepts are not limited with regard to the type of sensor(s) that is used for this purpose. This step can also be performed manually instead of automatically based on the signals output by the sensor(s). For example, an observer or the human subject can press a button to identify when the subject stepped off the beam 100. The algorithm 140 would then record the number of step-offs and when they occurred. In other embodiments, the output of the sensor(s) is processed to detect the number of step-offs and when they occurred. For example, capacitance sensors can be either attached to the soles of the shoes or located inside of the shoes worn by the human subject to measure when ground contact occurs to thereby detect step-offs. The capacitance sensors or various types of transducer sensors could instead also be located on the treadmill 101 on either side of the beam 100. As another alternative, inertial measurement units can be secured to the subject's ankle or foot for measuring ground contact. Optical sensors may also be used for this purpose. For example, a camera could be used to monitor step-offs by the subject. The type of sensor that is used can depend in large part on the type of human balance training course that is used. The sensor(s) can be, for example, touch or pressure sensor(s), inertial sensor(s), magnetic sensor(s), optical sensors. Sensors can also be used to record additional balance indicators in addition to ground contact/step offs to evaluate performance. Some examples of these additional balance performance indicators include center of mass displacement, center of mass velocity, and mediolateral sway, but there are other balance indicators that can be used.

Block 184 represents a processor performing a balance training algorithm that processes the output signals to record and/or assess the balance training performance of the human subject. In some embodiments, the balance training performance of the human subject is recorded for assessment at a later time. In other embodiments, the balance training performance is recorded and assessed. An example of the later case is recording the balance training performance in the current balance training session and assessing it by comparing it to balance training performance recorded in a previous balance training session. Another type of assessment can be, for example, comparing the recorded balance training performance for the current training session with the recorded pre-test performance from the pre-test that was performed earlier that day.

In accordance with a representative embodiment, the balance training algorithm 140 performed by the processor 120 counts the number of step-offs per minute during the balance training session and records the performance. In this embodiment, the number of step-offs was used as the balance performance indicator, but in other embodiments other balance performance indicators such as those mentioned above are used (e.g., center of mass displacement and velocity, and mediolateral sway, etc.).

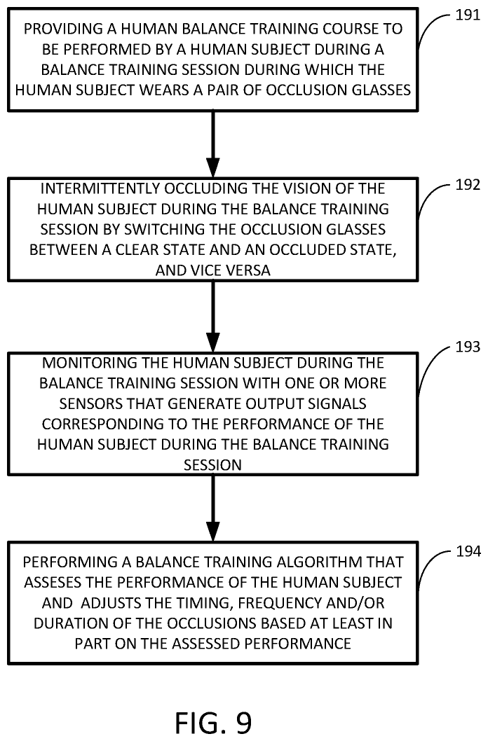

FIG. 9 is a flow diagram of the method performed by the data processing unit 110 running the algorithm 140 in accordance with another representative embodiment. Blocks 191-193 of FIG. 9 can be identical to blocks 181-183, respectively, of FIG. 8. In accordance with an embodiment, the balance training algorithm 140 controls the occlusion glasses 102 to adjust the timing, frequency and/or duration of the occlusions, i.e., the switching between the clear state and the occluded state, based at least in part on the assessed balance training performance. This is represented by block 194. In accordance with this representative embodiment, the balance training algorithm 140 processes the output signals generated by the sensor(s) and controls the timing, frequency and/or duration of the occlusions based at least in part on the output signals of the sensor(s). For example, assuming the balance performance indicator being used to assess performance is the number of step-offs, the algorithm 140 counts the number of step-offs per a given time interval (e.g., per minute) and reduces the timing, frequency and/or duration of the occluded state if the number of step-offs is above a predetermined threshold (TH) value. As with the embodiment represented by the flow diagram of FIG. 8, other balance performance indicators can be used to assess the performance, such as those mentioned above (e.g., center of mass displacement and velocity, and mediolateral sway, etc.).

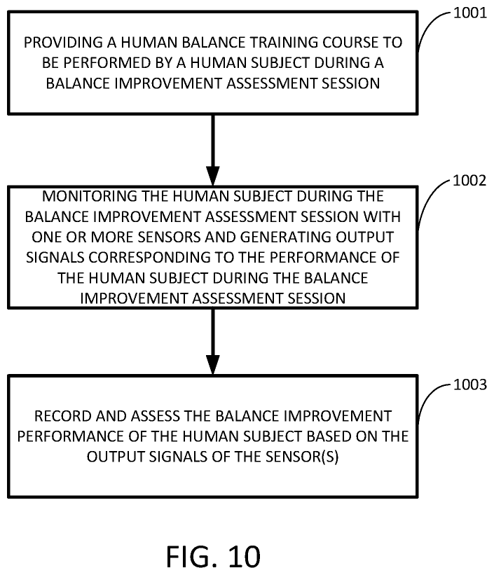

FIG. 10 is a flow diagram of the method in accordance with another representative embodiment. This embodiment can be performed as part of the pre-test, post-test or retention test assessments. Block 1001 represents the step of providing a human balance training course to be performed by a human subject during a pre-test, post-test or retention test balance improvement assessment session. The human subject preferably is either not wearing the occlusion glasses at all or is wearing the occlusion glasses with the glasses set to the clear state at all times during this session. Block 1002 represents monitoring the human subject during the balance improvement assessment session with one or more sensors that generate output signals corresponding to the performance by the human subject during the session. This step can be performed manually instead of automatically based on the signals output by the sensor(s). For example, as discussed above, an observer or the human subject can press a button to identify when the subject stepped off the beam 100. The algorithm 140 would then record the number of step-offs and when they occurred. In the preferred embodiment, the output of the sensor(s) is processed by the algorithm to detect the number of step-offs and when they occurred. The inventive principles and concepts are not limited with regard to the type of sensor(s) that is used for this purpose. For example, the capacitance sensors or the other types of sensors discussed above can be used for this purpose. Also, balance performance indicators other than step-offs can be monitored, including, for example, center of mass displacement, center of mass velocity, and mediolateral sway.

Block 1003 represents the balance improvement assessment algorithm, which can be part of the balance training algorithm 140 or separate from it, processing the output signals to record and assess the balance improvement performance of the human subject. Examples of the assessments include comparing the performance of the current post-test session with the recorded performance from an earlier pre-test session and comparing the retention test performance of the current session to pre-test and/or post-test performance recorded two weeks earlier for the same subject.

The flow diagrams of FIGS. 8, 9 and 10 show the functionality and operation of a possible implementation of one or more algorithms performed by the processor 120. Many variations may be made to the method represented by the flow diagrams while still achieving the goals described herein. Also, while various embodiments of the algorithm 140 have been described herein for carrying out specific tasks, other algorithms not specifically mentioned herein may be performed by the processor 120. For any or all portions of algorithm 140 that are implemented in software and/or firmware being executed by processor 120, the corresponding computer instructions can be stored in a non-transitory memory device, such as the memory device 130. For any component discussed herein that is implemented in the form of software or firmware, any one of a number of programming languages may be employed such as, for example, C, C++, C#, Objective C, Java®, JavaScript®, Perl, PRP, Visual Basic®, Python Ruby, Flash®, or other programming languages.

The memory device 120 can include, for example, random access memory (RAM), read-only memory (ROM), a hard drive, a solid-state drive, a USB flash drive, a memory card, an optical disc such as compact disc (CD) or digital versatile disc (DVD), a floppy disk, a magnetic tape, static random access memory (SRAM), dynamic random access memory (DRAM), magnetic random access memory (MRAM), a programmable read-only memory (PROM), an erasable programmable read-only memory (EPROM), an electrically erasable programmable read-only memory (EEPROM), or other like memory device.

It should be emphasized that the above-described embodiments of the present invention are merely possible examples of implementations, merely set forth for a clear understanding of the inventive principles and concepts. Many variations and modifications may be made to the above-described embodiments without departing from the scope of the present disclosure. All such modifications and variations are intended to be included herein within the scope of this disclosure and protected by the following claims.

1. A system for training balance in a human subject, the system comprising:
a human balance training course to be performed by the human subject during a balance training session;
a pair of occlusion glasses to be worn by the human subject during the balance training session, the pair of occlusion glasses being configured to intermittently occlude vision of the human subject as the human subject performs the human balance training course;
one or more sensors configured to monitor the human subject as the human subject performs the human balance training course, said one or more sensors generating output signals corresponding to the performance by the human subject of the human balance training course;
a memory device; and
a processor configured to perform a balance training algorithm that processes the output signals to record the performance of the human subject in the memory device.

2. The system of claim 1, wherein the balance training algorithm processes the output signals to assess the performance of the human subject.

3. The system of claim 2, wherein the balance training algorithm adjusts one or more of a timing, frequency and duration of the intermittent occlusions based at least in part on the assessment of the performance of the human subject.

4. The system of claim 2, wherein the balance training algorithm records the assessment of the performance of the human subject in the memory device.

5. The system of claim 4, wherein the balance training algorithm compares the recorded assessment with a previously recorded assessment of a previous performance by the human subject of the balance training course to determine whether the balance of the human subject has improved.

6. The system of claim 5, wherein the previously recorded assessment was obtained from output signals generated by said one or more sensors monitoring the previous performance by the human subject of the balance training course while not wearing the pair of occlusion glasses and not being subjected to intermittently occluded vision.

7. The system of claim 5, wherein the previously recorded assessment was obtained from output signals generated by said one or more sensors monitoring the previous performance by the human subject of the balance training course while wearing the pair of occlusion glasses and being subjected to intermittently occluded vision.

8. The system of claim 1, wherein the pair of occlusion glasses are incorporated into a housing in which the processor and memory device are housed, the housing comprising a user interface that has one or more controls that allow at least one of a timing, frequency and duration of the intermittent occlusions to be set and adjusted by a user.

9. A method for training balance in a human subject, the method comprising:
providing a human balance training course to be performed by the human subject during a balance training session during which the human subject wears a pair of occlusion glasses during the balance training session;
intermittently occluding vision of the human subject during the balance training session by switching the pair of occlusion glasses between a clear state in which the vision of the human subject is unoccluded and an occluded state in which the vision of the human subject is occluded, and vice versa;
with one or more sensors, monitoring the human subject during the balance training session and generating output signals corresponding to the performance by the human subject during the balance training session; and
with a processor, performing a balance training algorithm that processes the output signals and records the performance of the human subject in a memory device.

10. The method of claim 9, further comprising:
with the processor performing the balance training algorithm, processing the output signals to assess the performance of the human subject.

11. The method of claim 10, further comprising:
with the processor performing the balance training algorithm, causing one or more of a timing, frequency and duration of the intermittent occlusions to be adjusted based at least in part on the assessment of the performance of the human subject.

12. The method of claim 10, further comprising:
with the processor performing the balance training algorithm, recording the assessment of the performance of the human subject in the memory device.

13. The method of claim 12, further comprising:
with the processor performing the balance training algorithm, comparing the recorded assessment with a previously recorded assessment of a previous performance by the human subject of the balance training course to determine whether the balance of the human subject has improved.

14. The method of claim 13, wherein the previously recorded assessment was obtained from output signals generated by said one or more sensors monitoring the previous performance by the human subject of the balance training course while not wearing the pair of occlusion glasses and not being subjected to intermittently occluded vision.

15. The method of claim 13, wherein the previously recorded assessment was obtained from output signals generated by said one or more sensors monitoring the previous performance by the human subject of the balance training course while wearing the pair of occlusion glasses and being subjected to intermittently occluded vision.

16. The method of claim 9, wherein the pair of occlusion glasses are incorporated into a housing in which the processor and memory device are housed, the housing comprising a user interface that has one or more controls that allow at least one of a timing, frequency and duration of the intermittent occlusions to be set and adjusted by a user.

17. A computer program comprising computer instructions for execution by a processor, the computer instructions being embodied on a non-transitory computer-readable medium, the computer instructions comprising:
a first set of computer instructions for receiving output signals output from one or more sensors that monitor a performance by a human subject of a balance training course during a balance training session during which the human subject wears a pair of occlusion glasses that intermittently occlude vision of the human subject during the balance training session by switching the pair of occlusion glasses between a clear state in which the vision of the human subject is unoccluded and an occluded state in which the vision of the human subject is occluded, and vice versa; and
a second set of computer instructions that process the output signals to record the performance of the human subject in a memory device.

18. The computer program of claim 18, further comprising:
a third set of computer instructions that process the output signals to assess the performance of the human subject.

19. The computer program of claim 18, further comprising:
a fourth set of computer instructions that cause one or more of a timing, frequency and duration of the intermittent occlusions to be adjusted based at least in part on the assessment of the performance of the human subject.

20. The computer program of claim 18, further comprising:
a fifth set of computer instructions that compare the recorded assessment with a previously recorded assessment of a previous performance by the human subject of the balance training course to determine whether the balance of the human subject has improved.
